(19) United States
(12) Patent Application Publication
(10) Pub. No.: US 2014/0158589 A1
(43) Pub. Date: Jun. 12, 2014

(54) Blood Purification Apparatus

(71) Applicant: Nikkiso Company Limited, Tokyo (JP)

(72) Inventors: Tomohiro FURUHASHI, Makinohara-shi (JP); Akira SUGIOKA, Makinohara-shi (JP); Masahiro TOYODA, Makinohara-shi (JP)

(21) Appl. No.: 14/181,638

(22) Filed: Feb. 15, 2014

Related U.S. Application Data
(63) Continuation of application No. PCT/JP2012/070545, filed on Aug. 10, 2012.

(30) Foreign Application Priority Data
Aug. 17, 2011 (JP) 2011-178214

(51) Int. Cl. A61M 1/36; A61M 1/16

(52) U.S. Cl. CPC A61M 1/3626; A61M 1/367; A61M 1/1601
USPC 210/97; 210/198.1; 210/199

(57) ABSTRACT

A blood purification apparatus has a blood circuit with an arterial blood circuit (1) and a venous blood circuit (2). Blood is extracorporeally circulated from a tip of the arterial blood circuit (1) to a tip of the venous blood circuit (2). A dialyzer (3) is interposed between the arterial blood circuit (1) and the venous blood circuit (2) to purify the blood. The blood purification apparatus can substitute the blood inside the blood circuit during returning of the blood after treatment. An air circulation line is connected to a predetermined portion in the blood circuit to circulate air. An air pump (18) supplies the air into the blood circuit, via the air circulation line. A control device (19) controls the air pump (18) to supply the air into the blood circuit during returning of the blood.

### CROSS-REFERENCE TO RELATED APPLICATIONS

This application is a continuation of International Application No. PCT/JP2012/070545, filed Aug. 10, 2012, which claims priority to Japanese Application No. 2011-178214, filed Aug. 17, 2011. The disclosures of the above applications are incorporating herein by reference.

### FIELD

The present disclosure relates to a blood purification apparatus for extracorporeally circulating blood of a patient to purify the blood during dialysis treatment using a dialyzer.

### BACKGROUND

A hemodialysis apparatus as a blood purification apparatus, is mainly configured to include a blood circuit with an arterial blood circuit and a venous blood circuit. The arterial blood circuit has an arterial puncture needle. The venous blood circuit has a venous puncture needle. A dialyzer is interposed between the arterial blood circuit and the venous blood circuit to purify blood flowing in the blood circuit. A blood pump is arranged in the arterial blood circuit. An artery side air trap chamber and a vein side air trap chamber are, respectively, arranged in the arterial blood circuit and the venous blood circuit. A dialysis device supplies a dialysate to the dialyzer.

In addition, a containing device (so-called saline bag), containing a physiological saline solution, is connected between a tip in the arterial blood circuit and the blood pump via a physiological saline solution supplying line. This enables cleaning/priming before dialysis treatment. Also, it enables substitution during the dialysis treatment and returning of blood after the dialysis treatment. For example, during returning of the blood, the physiological saline solution inside the containing device is supplied into the blood circuit via the physiological saline solution supplying line. The blood inside the blood circuit is substituted with the physiological saline solution. This enables the blood to reflow into a body of a patient. For example, Japanese Unexamined Patent Application Publication No. 2004-222884 discloses returning blood work of a dialysis apparatus in the related art.

### SUMMARY

However, the above-described blood purification apparatus in the related art has the following problem.

During return of the blood after treatment, returning of the blood is supposed to be completed, in theory, if a substitution solution (physiological saline solution) is supplied to the blood circuit in an amount equivalent to an extracorporeally circulating amount of blood in the blood circuit (total capacity of the blood circuit and the air trap chambers). However, in practice, the substitution solution with the extracorporeally circulating amount or more is needed. This is due to the fact that if there is a stay portion in the flow route of the blood, such as the blood circuit and the air trap chambers, the blood (particularly, blood corpuscle constituent in the blood) is caused to stay in the stay portion and smooth substitution is hindered.

Thus, in order to more reliably substitute the blood with the substitution solution by supplying a large amount of the substitution solution to the blood circuit to return the blood increases treatment costs. Costs are increased due to an increased amount of the substitution solution to be used and the working hours for returning the blood are prolonged. Thus, this causes a heavy burden on the patient. Additionally, when blood is returned using a large amount of the substitution solution in the blood circuit, a problem exists in that a large amount of the substitution solution is introduced into the body of the patient together with the blood.

The present disclosure is made in view of such circumstances. The present disclosure aims to provide a blood purification apparatus that can reliably substitute blood during returning of the blood and can suppress a supply amount of the substitution solution.

According to the disclosure, a blood purification apparatus includes a blood circuit. The blood circuit includes an arterial blood circuit and a venous blood circuit. The blood circuit extracorporeally circulates blood of a patient from a tip of the arterial blood circuit to a tip of the venous blood circuit. A blood purification device is interposed between the arterial blood circuit and the venous blood circuit in the blood circuit. The blood purification device purifies the blood flowing in the blood circuit. The blood purification apparatus returns blood by substituting the blood inside the blood circuit after treatment. The blood purification apparatus further includes an air circulation line. The air circulation line is connected to a predetermined portion in the blood circuit to circulate air. During returning of the blood, the air can be supplied into the blood circuit via the air circulation line.

The blood purification apparatus further includes an air supplying device. The air supply device supplies air into the blood circuit via the air circulation line. A control device controls the air supplying device, which supplies air into the blood circuit during return of the blood.

The blood purification apparatus further includes an air trap chamber. The air trap chamber is connected to the arterial blood circuit or the venous blood circuit. The air can be supplied by connecting the air circulation line to the air trap chamber.

The blood purification apparatus further includes a liquid surface detection sensor. The sensor detects a liquid surface of the air trap chamber. When the liquid surface sensor detects the liquid surface, the air circulation line stops supplying the air.

The air supplying device supplies the air to or discharges air from the air trap chamber. The air supplying device can adjust the liquid surface level inside the air trap chamber.

The blood purification apparatus includes an air bubble detection sensor. The air bubble detection sensor detects an air bubble inside a flow route of a tip portion of the arterial blood circuit and the venous blood circuit. A valve device arranged in the tip portion can open and close the flow route of the tip portion. During returning of the blood, when the air bubble detection sensor detects an air bubble, the valve device is in a closed state and the air circulation line stops supplying the air.

The blood purification apparatus further includes a containing device. The containing device contains a predetermined amount of a physiological saline solution as a substitution solution. A substitution solution supplying device has a physiological saline solution supplying line that supplies the physiological saline solution inside the containing device into the blood circuit. The supplying line connects the containing device and a predetermined portion of the blood circuit to each other. Also, the substitution solution supplying device supplies the substitution solution into the blood circuit.

In the blood purification apparatus, the blood purification device back-filters a dialysate, as the substitution solution, and supplies the filtered dialysate into the blood circuit.

The air circulation line supplies air into the blood circuit during returning of the blood. Therefore, it is possible to reduce the amount of the blood to be substituted with the substitution solution by substituting the blood with the supplied air. Thus, it is possible to reliably substitute blood during returning of the blood, and it is possible to suppress the supply amount of the substitution solution.

The blood purification apparatus includes the air supplying device. The air supplying device supplies the air into the blood circuit via the air circulation line. The control device controls the air supplying device to supply the air into the blood circuit during returning of the blood. Therefore, it is possible to more reliably and accurately supply the air into the blood circuit during returning of the blood.

The blood purification apparatus includes the air trap chamber. The air trap chamber is connected to the arterial blood circuit or the venous blood circuit. The air can be supplied by connecting the air circulation line to the air trap chamber. Therefore, it is possible to substitute the blood inside the air trap chamber, that has a relatively large capacity and is likely to form a stay portion, with the air. Thus, it is possible to more reliably substitute the blood with the substitution solution during returning of the blood. Also, it is possible to suppress the supply amount of the substitution solution.

The blood purification apparatus includes the liquid surface detection sensor. The liquid surface detection sensor detects the liquid surface of the air trap chamber. When the liquid surface sensor detects the liquid surface, the air circulation line stops supplying air. Therefore, it is possible to suppress the supply amount of the substitution solution by at least a change amount of the liquid surface.

The air supplying device supplies the air to or discharges the air from the air trap chamber. This adjusts the liquid surface inside the air trap chamber. Therefore, in addition to the function of supplying the air during returning of the blood after the treatment, it is possible to provide a function of adjusting the liquid surface in the air trap chamber before the treatment or during the treatment.

The air bubble detection sensor detects an air bubble inside the flow route of the tip portion of the arterial blood circuit and the venous blood circuit. The valve device, arranged in the tip portion, can open and close the flow route of the tip portion. During returning of the blood, when the air bubble detection sensor detects the air bubble, the valve device is in the closed state and the air circulation line stops supplying air. Therefore, it is not necessary to perform the substitution by supplying the substitution solution into the blood circuit during returning of the blood.

The blood purification apparatus includes the containing device. The containing device contains the predetermined amount of the physiological saline solution as the substitution solution. The substitution solution supplying device has the physiological saline solution supplying line that can supply the physiological saline solution inside the containing device into the blood circuit. The supply line connects the containing device and the predetermined portion of the blood circuit to each other. The supply line supplies the substitution solution into the blood circuit. Therefore, it is possible to perform returning of the blood by adopting a general returning blood method of using the physiological saline solution as the substitution solution.

The blood purification device can back-filter the dialysate, as the substitution solution, and supply the filtered dialysate into the blood circuit. Therefore, it is possible to eliminate a need for a dedicated pipe to supply the substitution solution. Thus, it is possible to simplify the pipe.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic view of a blood purification apparatus, starting returning of blood after treatment, according to a first embodiment.

FIG. 2 is a schematic view of the blood purification apparatus where air and a substitution solution are supplied during returning of the blood.

FIG. 3 is a schematic view of the blood purification apparatus where the substitution solution is supplied during returning of the blood.

FIG. 4 is a schematic view of a blood purification apparatus, where air and a substitution solution are supplied during returning of blood, according to another embodiment.

FIG. 5 is a schematic view of a blood purification apparatus, where returning of blood is performed using back-filtration, according to a second embodiment.

FIG. 6 is a schematic view of an artery side air trap chamber in another embodiment which is applied to the blood purification apparatus.

FIG. 7 is a schematic view of a blood purification apparatus, where air and a substitution solution are supplied during returning of blood, according to a third embodiment.

### DETAILED DESCRIPTION

Hereinafter, an embodiment of the present disclosure will be described in detail with reference to the drawings.

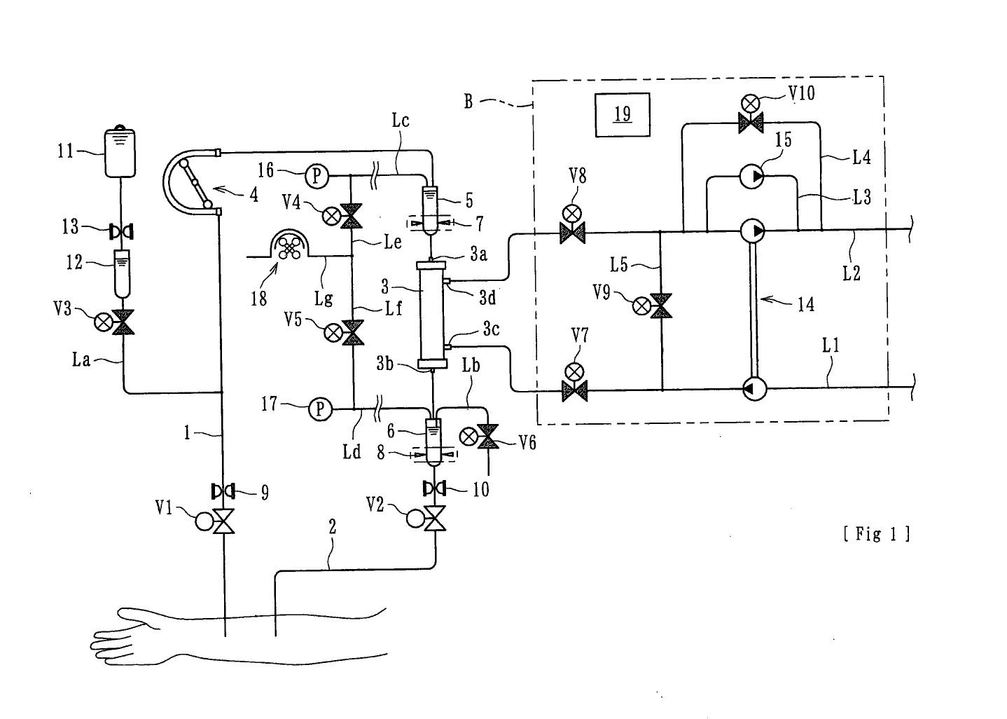

A blood purification apparatus according to a first embodiment has a hemodialysis apparatus performing hemodialysis treatment. In FIG. 1, it is mainly configured to include a blood circuit that includes an arterial blood circuit 1 and a venous blood circuit 2. A dialyzer 3 (blood purification device) is interposed between the arterial blood circuit 1 and the venous blood circuit 2. The dialyzer 3 purifies blood flowing in the blood circuit. A peristaltic blood pump 4 is arranged in the arterial blood circuit 1. An artery side air trap chamber 5 and a vein side air trap chamber 6 are, respectively, connected to an intermediate portion of the arterial blood circuit 1 and the venous blood circuit 2. A dialysis device B can supply a dialysate to the dialyzer 3. A control device 19 is arranged inside the dialysis device B.

In the arterial blood circuit 1, an arterial puncture needle is connected to its tip. The peristaltic blood pump 4 and the artery side air trap chamber 5, for removing air bubbles, are arranged in an intermediate portion of the arterial blood circuit 1. In contrast, in the venous blood circuit 2, a venous puncture needle is connected to its tip. The vein side air trap chamber 6, for removing air bubbles, is connected to an intermediate portion of the venous blood circuit 2. In addition, a physiological saline solution supplying line La extends from a containing device 11. The supplying line La is connected to a predetermined portion, a portion between an air bubble detection sensor 9 and the blood pump 4, of the arterial blood circuit 1.

The containing device 11 is a so-called “saline bag”. It contains a predetermined amount of a physiological saline solution as a substitution solution. The physiological saline solution supplying line La supplies the physiological saline solution inside the containing device 11 to the blood circuit. The supplying line connects the containing device 11 and a predetermined portion of the blood circuit, a portion between the air bubble detection sensor 9 and the blood pump 4, to each other. The containing device 11 and the physiological saline solution supplying line La form a “substitution solution supplying device”. The substitution solution supplying device supplies the substitution solution into the blood circuit. An electromagnetic valve V3, arranged in the physiological saline solution supplying line La, can arbitrarily open and close the flow route. An air bubble detection sensor 13 (liquid shortage sensor) is connected in the supplying line in order to detect a liquid level in an air trap chamber 12 and the containing device 11.

The artery side air trap chamber 5 and the vein side air trap chamber 6 can remove air bubbles by capturing the air bubbles in the blood inside the blood circuit. A filtration net (not illustrated) is arranged inside the trap chambers and can capture a thrombus, for example, during returning of blood. Monitor tubes Lc and Ld, respectively, extend from each upper portion (air layer side) of the artery side air trap chamber 5 and the vein side air trap chamber 6. The tips of the tubes Lc and Ld are, respectively, connected to pressure sensors 16 and 17. The monitor tubes Lc and Ld and the pressure sensors 16 and 17 can respectively measure a liquid pressure inside the artery side air trap chamber 5 (dialyzer inlet pressure) and a liquid pressure inside the vein side air trap chamber 6 (venous pressure).

The artery side air trap chamber 5 and the vein side air trap chamber 6 have liquid surface detection sensors 7 and 8 that can detect each liquid surface. The liquid surface detection sensors 7 and 8 are configured to have a sensor that can detect when the liquid surface is lowered down to a lower portion of the artery side air trap chamber 5 or the vein side air trap chamber 6. In addition, an overflow line Lb extends from an upper portion of the vein side air trap chamber 6. An electromagnetic valve V6 arranged in an intermediate portion of the overflow line Lb can arbitrarily open and close the overflow line Lb

In a tip portion of the arterial blood circuit 1 (vicinity of the arterial puncture needle), the air bubble detection sensor 9 can detect air bubbles inside the flow route of the tip portion. An electromagnetic valve V1, as a valve device, is arranged in the line and can open and close the flow route of the tip portion. The electromagnetic valve V1 is in the vicinity of an upstream side of the air bubble detection sensor 9. In a tip portion of the venous blood circuit 2, in the vicinity of the venous puncture needle, an air bubble detection sensor 10 is located to detect air bubbles inside a flow route of the tip portion. An electromagnetic valve V2, as a valve device, is arranged in the line and can open and close the flow route of the tip portion. The electromagnetic valve V2 is in the vicinity of an upstream side of the air bubble detection sensor 10. The air bubble detection sensors 9 and 10 and the electromagnetic valves V1 and V2 are generally fixed to the dialysis device B. However, they may be in a separate installation type, for example, a clip style so as to be respectively installed at any position in the arterial blood circuit 1 and the venous blood circuit 2.

If the blood pump 4 is rotated when the arterial puncture needle and the venous puncture needle are punctured into a patient, the blood of the patient reaches the dialyzer 3 through the arterial blood circuit 1. The blood is then purified by the dialyzer 3. The blood reflows into the body of the patient through the venous blood circuit 2. Air bubbles are removed in the vein side air trap chamber 6. That is, the blood of the patient is purified by the dialyzer 3 while the blood is extracorporeally circulated from the tip of the arterial blood circuit 1 to the tip of the venous blood circuit 2 in the blood circuit.

In the dialyzer 3, a housing unit has a blood inlet port 3a, a blood outlet port 3b, a dialysate inlet port 3c and a dialysate outlet port 3d. Among them, the arterial blood circuit 1 is connected to the blood inlet port 3a. The venous blood circuit 2 is connected to the blood outlet port 3b. In addition, the dialysate inlet port 3c and the dialysate outlet port 3d are, respectively, connected to a dialysate introduction line L1 and a dialysate discharge line L2. Each of the lines L1, L2 extends from the dialysis device B.

The dialyzer 3 accommodates a plurality of hollow fibers in its inside. An interior of the hollow fibers serves as the flow route for the blood. A space between an outer peripheral surface of the hollow fibers and an inner peripheral surface of the housing unit serves as the flow route of the dialysate. The hollow fiber has a hollow fiber membrane by forming multiple minute holes (pores) penetrating the outer peripheral surface and the inner peripheral surface. In this configuration, impurities in the blood can be transmitted into the dialysate via the membrane.

The dialysis device B has a duplex pump 14, ultrafiltration pump 15 and control device 19. The duplex pump is arranged across the dialysate introduction line L1 and the dialysate discharge line L2. The ultrafiltration pump 15 removes water from the patient's blood flowing in the dialyzer 3. One end of the dialyzer introduction line L1 is connected to the dialyzer 3 (dialysate inlet port 3c). The other end is connected to a dialysate supplying apparatus (not illustrated) that produces the dialysate with a predetermined concentration. In addition, one end of the dialysate discharge line L2 is connected to the dialyzer 3 (dialysate outlet port 3d). The other end is connected to a liquid discharge device (not illustrated). Rotation of the duplex pump 14 causes the dialysate, supplied from the dialysate supplying apparatus, to reach the dialyzer 3 through the dialysate introduction line L1. The dialysate is sent to the liquid discharge device through the dialysate discharge line L2.

A bypass flow route L3, which bypasses a liquid discharge side of the duplex pump 14, extends to the dialysate discharge line L2. The ultrafiltration pump 15 is arranged in an intermediate portion of the bypass flow route L3. A bypass flow route L4, which bypasses the duplex pump 14 and the bypass flow route L3, extends to the dialysate discharge line L2. An electromagnetic valve V10 can arbitrarily open and close the flow route. The electromagnetic valve V10 is arranged in the intermediate portion of the bypass flow route L4. A reference numeral L5, in the drawing, represents a bypass flow route that connects the dialysate introduction line L1 and the dialysate discharge line L2 to each other and bypasses the dialyzer 3. An electromagnetic valve V9, that can arbitrarily open and close the flow route, is arranged in the bypass flow route L5.

The present embodiment includes air circulation lines (Le, Lf and Lg). The air flows through the lines and are connected to a predetermined portion in the blood circuit. An air pump 18 (air supplying device) supplies the air into the blood circuit via the air circulation lines (Le, Lf and Lg). A control device 19 controls the air pump 18 to supply the air into the blood circuit during returning of blood. For convenience of layout in the drawing, pressure sensors 16 and 17 and the air pump 18, as the air supplying device, are illustrated as if they are located in a position different from that of the dialysis device B, but are actually arranged in the dialysis device B.

The air circulation line includes a flexible tube that can circulate the air. The air circulation line is mainly configured to have the flow route Le. The tip of Le is connected to the intermediate portion of the monitor tube Lc. The flow route Lf has a tip connected to the intermediate portion of the monitor tube Ld. The flow route Lg has tips, respectively, connected to base ends of the flow routes Le and Lf. The base end of Lg is exposed to air. That is, the tip of the flow route Lg is divided into the flow routes Le and Lf. The respective tips of the flow routes Le and Lf are connected to the intermediate portions of the monitor tubes Lc and Ld.

In this manner, the flow routes Lg and Le, configuring the air circulation line, are connected to an upper portion of the artery side air trap chamber 5, via the monitor tube Lc. The flow routes Lg and Lf, similarly configuring the air circulation line, are connected to an upper portion of the vein side air trap chamber 6, via the monitor tube Ld. In addition, an electromagnetic valve V4, that can arbitrarily open and close the flow route, is arranged in the intermediate portion of the flow route Le. An electromagnetic valve V5, that can arbitrarily open and close the flow route, is arranged in the intermediate portion of the flow route Lf.

The air pump 18 may be a peristaltic pump. The air pump 18 is arranged in the flow route Lg to configure the air circulation line. The air pump 18 can be rotated in a normal rotation, rotation to the right in the drawing, and in a reverse rotation, rotation to the left in the drawing. If the air pump 18 is in the normal rotation mode, the air is sucked into the base end of the flow route Lg. The air can be supplied into the artery side air trap chamber 5 or the vein side air trap chamber 6. In addition, if the air pump 18 is in the reverse rotation, air is sucked into the air layer side of the artery side air trap chamber 5 or the vein side air trap chamber 6. The air can be discharged by the base end of the flow route Lg.

In this manner, when the air pump 18 performs normal rotation, the liquid surface of the artery side air trap chamber 5 or the vein side air trap chamber 6 can be lowered. When the air pump 18 is in the reverse rotation, the liquid surface of the artery side air trap chamber 5 or the vein side air trap chamber 6 can be raised. Since the air pump 18, according to the present embodiment, can be in the normal rotation and in the reverse rotation, the air can be supplied to, during the normal rotation, or discharged from, during the reverse rotation, the artery side air trap chamber 5 or the vein side air trap chamber 6. In this configuration, it is possible to adjust the liquid surface inside the artery side air trap chamber 5 and the vein side air trap chamber 6.

The control device 19 may be a microcomputer arranged in the dialysis device B. The control device 19 is electrically connected to an actuator configuring this blood purification apparatus (blood pump 4, air pump 18, duplex pump 14, ultrafiltration pump 15 and the like), electromagnetic valves V1 to V10 and various sensors (liquid surface detection sensors 7 and 8, air bubble detection sensors 9 and 10, pressure sensors 16 and 17 and the like). The control device 19 can automatically control a priming operation before treatment, various treatment operations during treatment and returning of blood after treatment.

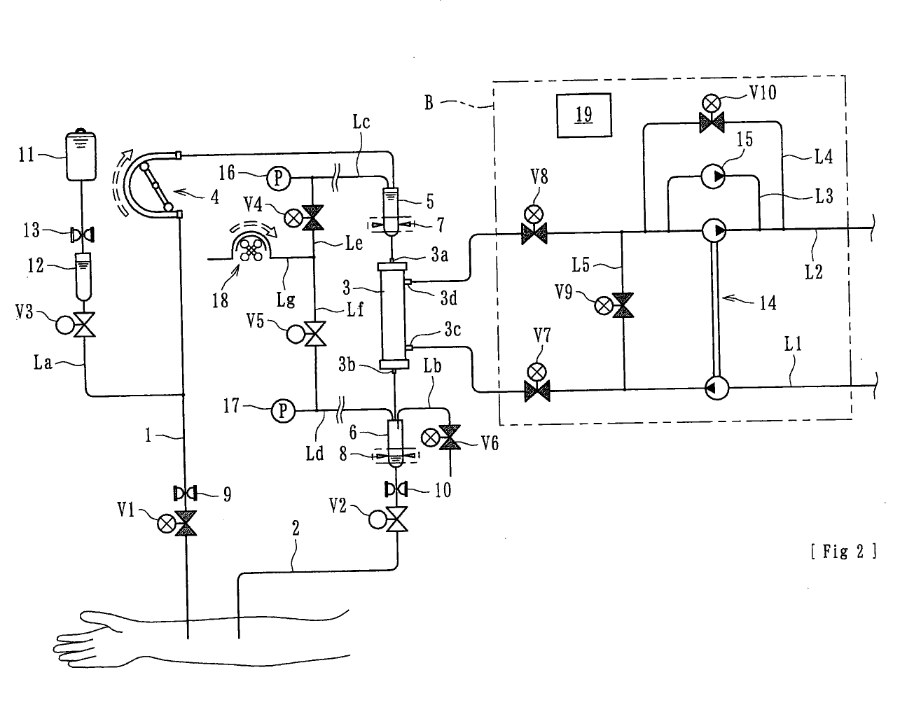

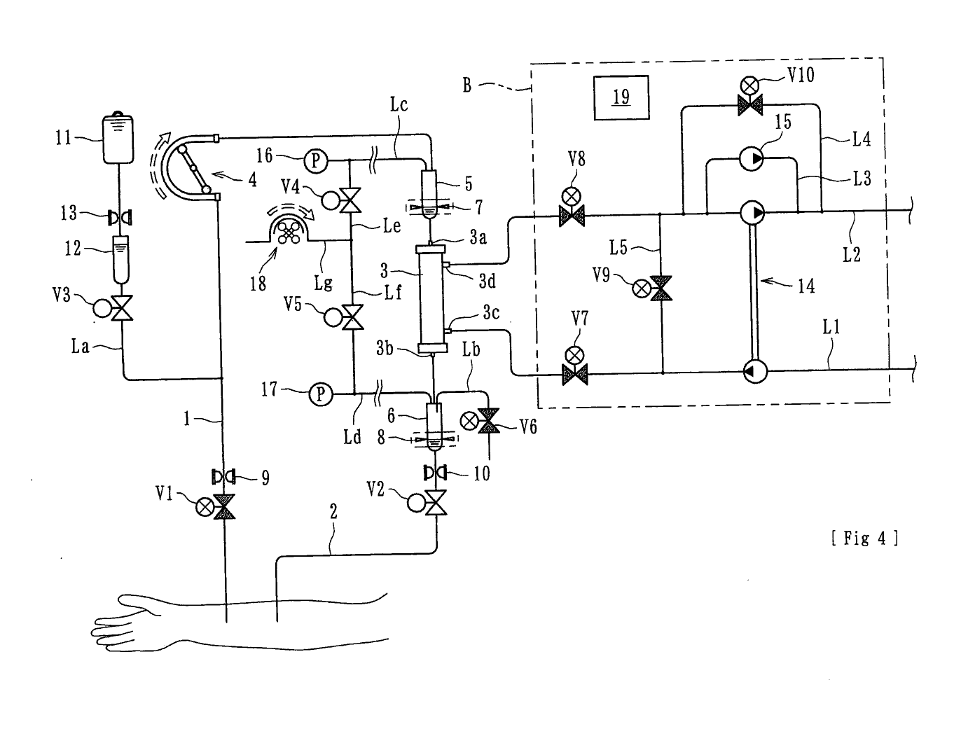

Thus, in addition to a series of controls relating to the treatment, the control device 19, according to the present embodiment, is capable of supplying the air into the blood circuit via the artery side air trap chamber 5 or the vein side air trap chamber 6. This is accomplished by controlling the rotation of the air pump 18 at a predetermined time during returning of the blood. For example, during returning of the blood, the control device 19 leaves the electromagnetic valve V4 in a closed state and the electromagnetic valve V5 in an opened state. The air pump 18 is rotated in the normal rotation (refer to FIG. 2) to enable the air to be supplied to the vein side air trap chamber 6, only. The control device 19 leaves the electromagnetic valves V4 and V5 in the opened state. The air pump 18 is rotated in the normal rotation (refer to FIG. 4) to enable the air to be supplied to both the artery side air trap chamber 5 and the vein side air trap chamber 6.

A control method during returning of the blood in the blood purification apparatus according to the present embodiment will be described.

As illustrated in FIG. 1, after the blood purification treatment is completed, the blood of the patient remains inside the blood circuit (arterial blood circuit 1 and venous blood circuit 2. The artery side air trap chamber 5 and the vein side air trap chamber 6 require returning of the blood. This requires the blood to reflow into the body of the patient. A process may be automatically conducted transited to a returning blood process after the treatment is completed. Alternatively, the process may be manually transited to the returning blood process after the treatment is completed. In addition, in the present embodiment, any one of the electromagnetic valves V1 and V2 may be in the opened state when starting returning of the blood, but the electromagnetic valves V1 and V2 may be in the closed state.

As illustrated in FIG. 2, when returning of the blood is started, the control device 19 leaves electromagnetic valve V1 in the closed state; leaves the electromagnetic valves V2 and V3 in the opened state; leaves the electromagnetic valve V4 in the closed state; and leaves the electromagnetic valve V5 in the opened state. At this time, the electromagnetic valves V6 to V10 are left in the closed state and the duplex pump 14 and the ultrafiltration pump 15 are left in a stopped state. However, the duplex pump 14 may be left in a rotated state. In this case, the electromagnetic valve V9 is left in the opened state. The control device 19 controls the blood pump 4 to be rotated in the normal rotation to supply the physiological saline solution (substitution solution), inside the containing device 11, to the blood circuit, via the physiological saline solution supplying line La. The control device 19 controls the air pump 18 to be in the normal rotation to supply the air to the vein side air trap chamber 6, via the flow routes Lg and Lf and the monitor tube Ld, that form the air circulation line.

In this manner, the blood inside the vein side air trap chamber 6 is substituted with the air and the liquid surface level is lowered. The blood inside the venous blood circuit 2, the blood in the further downstream side (blood pump 4 side) from a connection portion to the physiological saline solution supplying line La in the arterial blood circuit 1 and the blood inside the artery side air trap chamber 5 are substituted with the physiological saline solution. This enables return of the blood into the body of the patient. That is, during returning of the blood, it is possible to reduce the blood inside the vein side air trap chamber 6 by supplying air to the vein side air trap chamber 6 to lower the liquid surface level. Thus, it is possible to reduce the supply amount (use amount) of the physiological saline solution (substitution solution) to be substituted, by at least the reduced amount of the blood.

Thereafter, if the liquid surface detection sensor 8 detects the liquid surface (that is, if a state is detected where almost everything inside the vein side air trap chamber 6 is substituted with the air), the rotation of the air pump 18 is stopped. Also, the supply of the air is stopped. Then, when the blood pump 4 supplies a predetermined amount of the physiological saline solution or a blood discrimination device (not illustrated) disposed in the tip of the venous blood circuit 2 detects that the blood is substituted with the physiological saline solution, the rotation of the blood pump 4 is stopped to leave the electromagnetic valve V2 in the closed state. When the rotation of the air pump 18 is stopped, both of the electromagnetic valves V4 and V5 may be controlled to be left in the closed state.

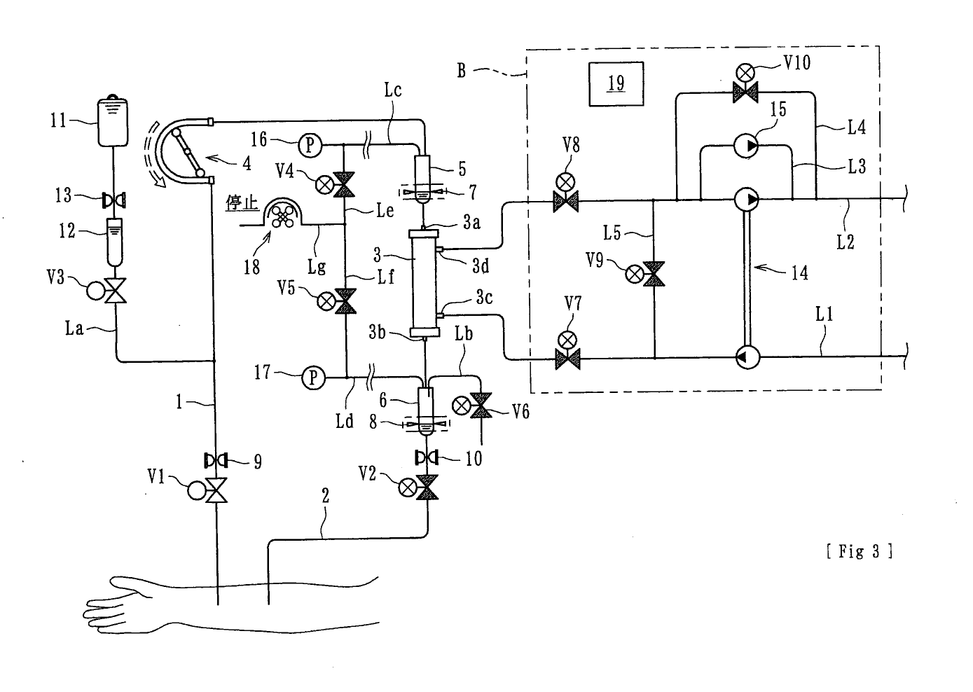

In this way, returning the blood of the venous blood circuit 2 side is completed. Subsequently, returning the blood of the arterial blood circuit 1 side is started. If returning the blood of the arterial blood circuit 1 side is started, the control device 19 controls the electromagnetic valves V1 and V2, so that they are in the closed state; controls the electromagnetic valve V3, to be in the opened state; and controls the blood pump 4, to be in the normal rotation. This enables the vein side air trap chamber 6 to store the physiological saline solution by an empty capacity (a capacity of the air layer after the liquid level is lowered). Then, as illustrated in FIG. 3, the control device 19 controls the blood pump 4 to rotate in the reverse rotation direction. This causes the physiological saline solution stored in the vein side air trap chamber 6 to flow into the arterial blood circuit 1 to perform returning of the blood. At this time, the electromagnetic valve V1 is left in the opened state and the electromagnetic valve V2 is left in the closed state. While maintaining the closed state of the electromagnetic valve V4, the electromagnetic valve V5 is left in the closed state. In this way, returning of the blood controlled by the control device 19 is completed.

Instead of the above-described manner, returning the blood of the arterial blood circuit 1 side can be performed in the following manner. That is, if returning the blood of the arterial blood circuit 1 side is started, the control device 19 controls the electromagnetic valve V2 to be in the closed state and controls the electromagnetic valves V1 and V3 to be in the opened state so as to maintain a stopped state of the blood pump 4. In this manner, self-weight of the physiological saline solution causes the physiological saline solution inside the containing device 11 to flow into the arterial blood circuit 1, via the physiological saline solution supplying line La, to be substituted with the blood.

Thus, an operation to lower the liquid surface level of the vein side air trap chamber 6, that is caused by the normal rotation of the air pump 18, is preferably completed at the latest until the physiological saline solution supplied by the rotation of the blood pump 4 reaches the vein side air trap chamber 6. In addition, in the present embodiment, the operation to lower the liquid surface of the vein side air trap chamber 6, that is caused by the normal rotation of the air pump 18, is performed until the liquid surface detection sensor 8 detects the liquid surfaces. However, instead of this manner, the operation may be set to be performed until the number of rotations or the rotation time of the air pump 18 reaches a predetermined value.

Here, the above-described embodiment is configured such that rotation of the air pump 18 enables the air to be supplied into the blood circuit via the air circulation lines (Le, Lf and Lg). However, instead of the air pump 18, an electromagnetic valve may be arranged in the line. In this case, for example, if the electromagnetic valve, arranged instead of the air pump 18, and the electromagnetic valve V4 or the electromagnetic valve V5 are left in the opened state and the electromagnetic valves V8 and V10 inside the dialysis device B are left in the opened state, the self-weight of the dialysate causes the dialysate inside the dialyzer 3 to flow to the dialysate discharge line L2 side. In this manner, it is possible to introduce the air from the tip of the air circulation line Lg and to supply the air into the blood circuit. That is, if the electromagnetic valves V8 and V10 are left in the opened state, the pipe of the dialysate inside the blood circuit and the dialysis device B is in an exposed state to the air. Then, potential energy based on a difference in a height between the artery side air trap chamber 5 or the vein side air trap chamber 6 and the electromagnetic valve V10 causes moisture of the blood in the blood circuit to be filtered. Thus, the blood moves to the dialysate discharge line L2 side. Therefore, it is possible to introduce the air from the tip of the air circulation line Lg and to supply the air to the artery side air trap chamber 5 or the vein side air trap chamber 6.

In the present embodiment, when starting returning of the blood, the air pump 18 is rotated and the blood pump 4 is in the normal rotation direction. However, instead of this manner, when starting returning of the blood, after the air pump 18 is rotated to supply the air into the vein side air trap chamber 6 and the blood inside the vein side air trap chamber 6 is substituted with the air (after the liquid surface detection sensor 8 detects the liquid surface), the blood pump 4 may be in the normal rotation or in the reverse rotation direction.

Furthermore, in the above-described embodiment, during returning of the blood, the air is supplied to the vein side air trap chamber 6 only. However, instead of this manner, as illustrated in FIG. 4, during returning of the blood, the control device 19 may control both of the electromagnetic valves V4 and V5 to be in the opened state and may control the air pump 18 to be in the normal rotation. In this case, it is possible to supply the air to both of the artery side air trap chamber 5 and the vein side air trap chamber 6. This occurs by rotation of the air pump 18 until the liquid surface detection sensors 7 and 8, respectively, detect the liquid surface.

According to the first embodiment as described above, during returning of the blood, the air pump 18 (air supplying device) is controlled to supply the air into the blood circuit. Therefore, it is possible to reduce the amount of the blood to be substituted with the physiological saline solution (substitution solution) by substituting the blood with the supplied air. Consequently, it is possible to reliably substitute the blood with the physiological saline solution during returning of the blood. Thus, it is possible to suppress the supply amount of the physiological saline solution.

In addition, the artery side air trap chamber 5 and the vein side air trap chamber 6 are, respectively, connected to the arterial blood circuit 1 and the venous blood circuit 2. The air circulation line is connected to the artery side air trap chamber 5 and the vein side air trap chamber 6. The air pump 18 can supply air to the air circulation line. Therefore, the blood inside the artery side air trap chamber 5 and the vein side air trap chamber 6, which have a relatively large capacity and are likely to form a stay portion, can be substituted with the air. Consequently, it is possible to more reliably substitute the blood with the physiological saline solution during returning of the blood. Thus, it is possible to suppress the supply amount of the physiological saline solution.

In addition, according to the present embodiment, the liquid surface detection sensor 8 can detect the liquid surface of the vein side air trap chamber 6. When the liquid surface detection sensor 8 detects the liquid surface level, the control device 19 stops the air pump 18 so that it does not supply air. Therefore, it is possible to suppress the supply amount of the physiological saline solution by the change amount of the liquid surface. According to another embodiment as illustrated in FIG. 4, when the liquid surface detection sensors 7 and 8 detect the liquid surface levels, the control device 19 stops the air pump 18 so that it does not supply air. Consequently, it is possible to suppress the supply amount of the physiological saline solution by at least a total changed amount of the liquid surface of the artery side air trap chamber 5 and the vein side air trap chamber 6.

Further, the air pump 18 can supply air to or discharge air from the artery side air trap chamber 5 and the vein side air trap chamber 6. Thus, the liquid surface level inside the artery side air trap chamber 5 and the vein side air trap chamber 6 can be adjusted. Therefore, in addition to the function of supplying the air during returning of the blood after treatment, it is possible to provide a function of adjusting the liquid surface level in the artery side air trap chamber 5 and the vein side air trap chamber 6 before the treatment or during the treatment.

The containing device 11 contains the predetermined amount of physiological saline solution as the substitution solution. The substitution solution supplying device has the physiological saline solution supplying line La. The line La connects the containing device 11 and the predetermined portion of the blood circuit to each other. The line La supplies the physiological saline solution inside the containing device 11 into the blood circuit. Therefore, it is possible to perform returning of the blood by adopting the general returning blood method by using the physiological saline solution as the substitution solution.

Next, a blood purification apparatus according to a second embodiment of the present disclosure will be described.

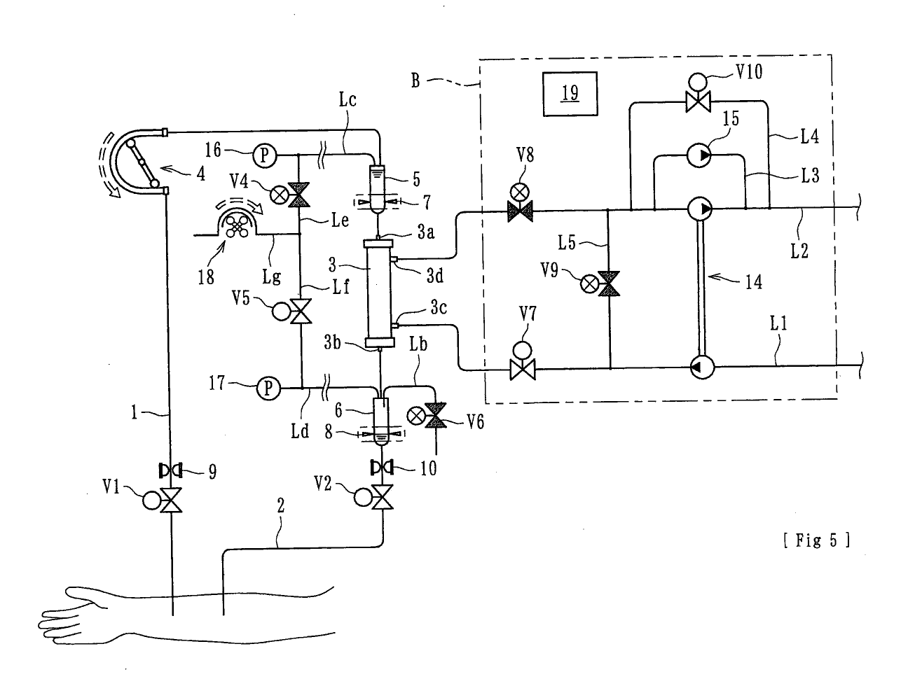

Similar to the first embodiment, the blood purification apparatus according to the present embodiment includes a hemodialysis apparatus to perform hemodialysis treatment. As illustrated in FIG. 5, it includes a blood circuit with the arterial blood circuit 1 and the venous blood circuit 2. The dialyzer 3 (blood purification device) is interposed between the arterial blood circuit 1 and the venous blood circuit 2. The dialyzer 3 purifies the blood flowing in the blood circuit. The peristaltic blood pump 4 is arranged in the arterial blood circuit 1. The artery side air trap chamber 5 and the vein side air trap chamber 6 are, respectively, connected to the intermediate portion of the arterial blood circuit 1 and the venous blood circuit 2. The dialysis device B can supply the dialysate to the dialyzer 3. The control device 19 is arranged inside the dialysis device B. The same reference numerals are given to the same components as those in the first embodiment, and their description will be omitted.

The dialyzer 3 (blood purification device) according to the present embodiment can perform back-filtration on the dialysate as the substitution solution. It supplies the filtered dialysate into the blood circuit. Specifically, as illustrated in FIG. 5, during returning of the blood, the control device 19 controls the electromagnetic valves V1 and V2 to be in the opened state; controls the electromagnetic valves V5, V7 and V10 to be in the opened state; and controls the electromagnetic valves V4, V8 and V9 to be in the closed state.

In such a state, the control device 19 rotates the air pump 18, the duplex pump 14 and the blood pump 4 (blood pump 4 is in the reverse rotation). At this time, the blood pump 4 is set to be in the reverse rotation at a speed slower than that of the duplex pump 14. Thus, the normal rotation of the air pump 18 enables the air to be supplied to the vein side air trap chamber 6. The rotation of the duplex pump 14 enables the dialysate to be supplied, by pumping, to the dialysate flow route of the dialyzer 3 via the dialysate introduction line L1. Thus, this performs the back-filtration in the blood circuit side. Since the blood pump 4 runs in the reverse rotation direction at a speed slower than that of the duplex pump 14, the dialysate subjected to the back-filtration flows into both of the arterial blood circuit 1 and the venous blood circuit 2. The dialysate is substituted with the blood in the respective blood circuits.

When the liquid surface detection sensor 8 detects the liquid surface, the rotation of the air pump 18 is stopped. The blood pump 4 supplies the predetermined amount of physiological saline solution. When the blood discrimination device (not illustrated) disposed at the tip portion of the arterial blood circuit 1 and the venous blood circuit 2 detects that the blood is substituted with the dialysate, the rotation of the blood pump 4 and the duplex pump 14 is stopped. The electromagnetic valves V1 and V2 are in the closed state. In this manner as described above, returning of the blood is completed.

Therefore, it is preferable that the air pump 18 is rotated at such a speed that the liquid surface is lowered until the dialysate reaches the vein side air trap chamber 6 at the latest. In addition, in the present embodiment, the operation to lower the liquid surface level of the vein side air trap chamber 6 by using the normal rotation of the air pump 18 is performed until the liquid surface detection sensor 8 detects the liquid surface. However, instead of this manner, the operation may be set to be performed until the number of rotations or the rotation time of the air pump 18 reaches a predetermined value.

In the present embodiment, when starting returning of the blood, the air pump 18 is rotated. The blood pump 4 and the duplex pump 14 are also rotated. However, instead of this manner, when starting returning of the blood, after the air pump 18 is rotated to supply the air into the vein side air trap chamber 6 and the blood inside the vein side air trap chamber 6 is substituted with the air (after the liquid surface detection sensor 8 detects the liquid surface), the blood pump 4 and the duplex pump 14 may be rotated.

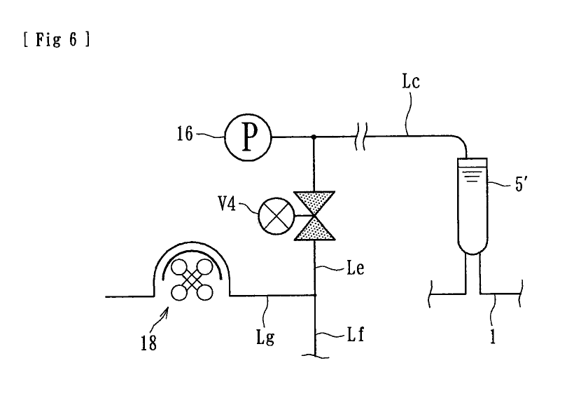

Furthermore, instead of the artery side air trap chamber 5, according to the present embodiment as illustrated in FIG. 6, it is preferable to adopt an artery side air trap chamber 5′. Here, an inlet port of the blood from the arterial blood circuit 1 and an outlet port of the blood to the arterial blood circuit 1 are formed in a lower portion of the air trap chamber. If such an artery side air trap chamber 5′ is adopted, it is possible to more reliably and smoothly substitute the blood inside the arterial blood circuit 1, with the dialysate as the substitution solution, and to perform returning of the blood.

According to the second embodiment as described above, similar to the first embodiment, during returning of the blood, the air pump 18 (air supplying device) is controlled to supply the air into the blood circuit. Therefore, it is possible to reduce the amount of the blood to be substituted with the dialysate (substitution solution) by substituting the blood with the supplied air. Consequently, it is possible to reliably substitute the blood with the dialysate during returning of the blood. Thus, it is possible to suppress the supply amount of the dialysate.

In addition, the artery side air trap chamber 5 and the vein side air trap chamber 6 are connected to the arterial blood circuit 1 and the venous blood circuit 2, respectively. The air circulation line is connected to the artery side air trap chamber 5 and the vein side air trap chamber 6. The air pump 18 supplies the air to the air circulation line. Therefore, the blood inside the artery side air trap chamber 5 and the vein side air trap chamber 6, that have relatively large capacity and are likely to form the stay portions, can be substituted with the air. Consequently, it is possible to more reliably substitute the blood with the dialysate during returning of the blood. Thus, it is possible to suppress the supply amount of the dialysate.

Further, the air pump 18 can supply air to or can discharge the air from artery side air trap chamber 5 and the vein side air trap chamber 6. Thus, the liquid surface inside the artery side air trap chamber 5 and the vein side air trap chamber 6 can be adjusted. Therefore, in addition to the function of supplying air during returning of the blood after the treatment, it is possible to provide the function of adjusting the liquid surface in the artery side air trap chamber 5 and the vein side air trap chamber 6 before the treatment or during the treatment.

In addition, according to the present embodiment, the liquid surface detection sensor 8 can detect the liquid surface of the vein side air trap chamber 6. When the liquid surface detection sensor 8 detects the liquid surface, the control device 19 stops the air pump 18 so that it does not supply air. Therefore, it is possible to suppress the supply amount of the dialysate by the changed amount of the liquid surface level. In particular, the dialyzer 3 (blood purification device), according to the present embodiment, can perform the back-filtration on the dialysate as the substitution solution. The dialyzer 3 supplies the filtered dialysate into the blood circuit. Therefore, it is possible to eliminate a need for a dedicated pipe, for example, the physiological saline solution supplying line La as described in the first embodiment, to supply the substitution solution. Thus, it is possible to simplify the pipe. Furthermore, according to the present embodiment, it is not necessary to use the physiological saline solution as the substitution solution during returning of the blood. Therefore, it is not necessary to provide the substitution solution supplying device with the containing device 11 and the physiological saline solution supplying line La as described in the first embodiment.

In the present embodiment, the dialyzer 3 can perform the back-filtration on the dialysate, as the substitution solution, and supply the filtered dialysate into the blood circuit. However, instead of this manner, a flexible tube may be connected between the dialysate supplying line L1 and the blood circuit, for example, a portion between the blood pump 4 and the electromagnetic valve V1 in the arterial blood circuit 1. The dialysate, as the substitution solution, may be supplied from the dialysate supplying line L1 to the blood circuit through the flexible tube. In addition, in this case, it is possible to substitute the blood with the dialysate by the duplex pump 14. Alternatively, a peristaltic dialysate substitution pump can be arranged in the flexible tube between the dialysate supplying line L1 and the blood circuit. The pump 14 is rotated and supplies the dialysate into the blood circuit.

Next, a blood purification apparatus according to a third embodiment will be described.

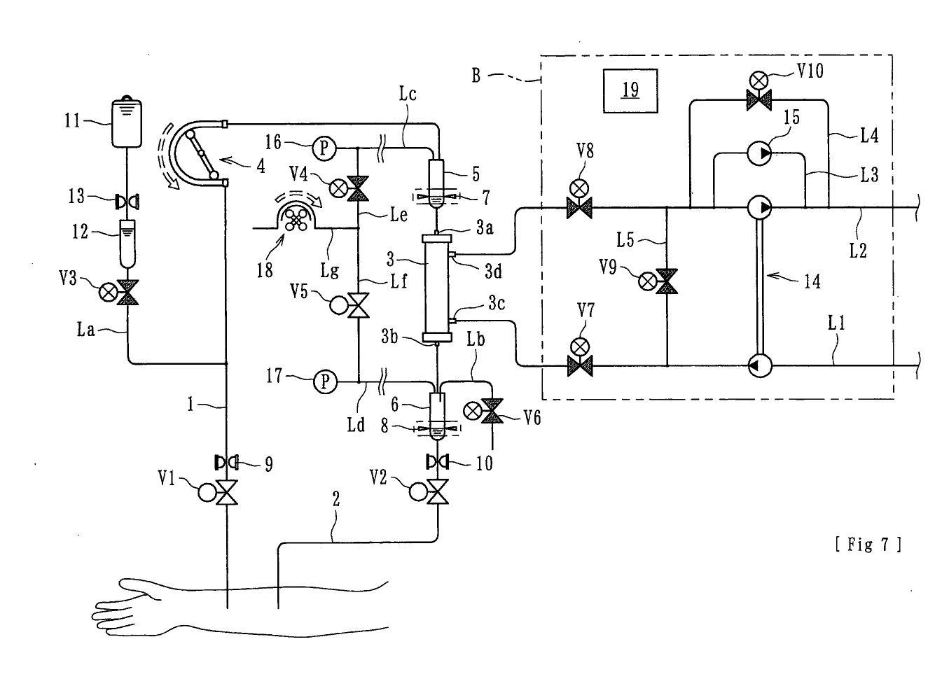

Similar to the first and second embodiments, the blood purification apparatus according to the present embodiment has a hemodialysis apparatus to perform hemodialysis treatment as illustrated in FIG. 7. It includes a blood circuit with the arterial blood circuit 1 and the venous blood circuit 2. The dialyzer 3 (blood purification device) is interposed between the arterial blood circuit 1 and the venous blood circuit 2. The dialyzer 3 purifies the blood flowing in the blood circuit. The peristaltic blood pump 4 is arranged in the arterial blood circuit 1. The artery side air trap chamber 5 and the vein side air trap chamber 6 are, respectively, connected to the intermediate portion of the arterial blood circuit 1 and the venous blood circuit 2. The dialysis device B can supply the dialysate to the dialyzer 3. The control device 19 is arranged inside the dialysis device B. The same reference numerals are given to components that are the same as those in the first and second embodiments, and their description will be omitted.

When the air bubble detection sensors 9 and 10 detect air bubbles during returning of the blood, the control device 19, according to the present embodiment, controls the electromagnetic valves V1 or V2. The valve devices V1, V2 are in the closed state. The control device 19 controls the air pump 18 to stop supplying the air. That is, similar to the first and second embodiments. Without using the substitution solution, such as the physiological saline solution or the dialysate, the blood is substituted with the air supplied from the air pump 18.

More specifically, as illustrated in FIG. 7, during returning of the blood after the treatment is completed, the control device 19 controls the electromagnetic valves V1, V2 and V5. The valves V1, V2 and V5 are in the opened state. The control device 19 controls the electromagnetic valves V3, V4 and V6 that are in the closed state. Thereafter, the blood pump 4 is moved into the reverse rotation and the air pump 18 is in the normal rotation. At this time, the blood pump 4 is in reverse rotation at a speed slower than that of the air pump 18. In this manner, the air supplied by the air pump 18 is set to be simultaneously supplied into both of the arterial blood circuit 1 and the venous blood circuit 2.

When the air bubble detection sensor 9 detects air bubbles, the electromagnetic valve V1 is left in the closed state. When the air bubble detection sensor 10 detects air bubbles, the electromagnetic valve V2 is left in the closed state. In this manner, returning of the blood can be simultaneously performed on the arterial blood circuit 1 and the venous blood circuit 2. Therefore, it is not necessary to substitute the blood by supplying the substitution solution (physiological saline solution or dialysate) to the blood circuit during returning of the blood.

Here, the above-described embodiment is configured such that rotation of the air pump 18 enables air to be supplied into the blood circuit via the air circulation lines (Le, Lf and Lg). However, for example, instead of the air pump 18, an electromagnetic valve may be arranged in the line. In this case, if the electromagnetic valve, arranged instead of the air pump 18, and the electromagnetic valve V4 or the electromagnetic valve V5 are left in the opened state and the electromagnetic valves V8 and V10, inside the dialysis device B, are left in the opened state, the self-weight of the dialysate causes the dialysate inside the dialyzer 3 to flow into the dialysate discharge line L2 side. In this manner, it is possible to introduce the air from the tip of the air circulation line Lg and supply the air into the blood circuit. That is, if the electromagnetic valves V8 and V10 are left in the opened state, the pipe of the dialysate inside the blood circuit and the dialysis device B is in an exposed state to the air. Then, potential energy based on a difference in a height between the artery side air trap chamber 5 or the vein side air trap chamber 6 and the electromagnetic valve V10 causes moisture of the blood in the blood circuit to be filtered. Thus, the blood is moved to the dialysate discharge line L2. Therefore, it is possible to introduce the air from the tip of the air circulation line Lg and to supply the air into the artery side air trap chamber 5 or the vein side air trap chamber 6.

In the above-described embodiment, the electromagnetic valve V4 is left in the closed state. The electromagnetic valve V5 is left in the opened state. This supplies the air via the vein side air trap chamber 6. However, instead of this manner, a configuration may be made such that the air is supplied via the artery side air trap chamber 5. Here, the electromagnetic valve V4 is left in the opened state and the electromagnetic valve V5 in the closed state. In addition, during returning of the blood, the rotation of the blood pump 4 and the air pump 18 may not be simultaneously started as compared to the present embodiment. For example, the air pump 18 may be first caused to be rotated and then the blood pump 4 may be rotated in the reverse rotation. Furthermore, as described above, returning of the blood according to the method of the first embodiment, for example, may be performed to some extent before returning of the blood according to the present embodiment may be performed. Thus, in the present embodiment, it is preferable to configure to substitute the blood of more patients with the air by installing the air bubble detection sensors 9 and 10 and the electromagnetic valves V1 and V2 such that the arrangement positions are close to the tip of the arterial blood circuit 1 or the tip of the venous blood circuit 2, vicinity of the arterial puncture needle or the venous puncture needle.

According to the third embodiment as described above, similar to the first and second embodiments, during returning of the blood, the air pump 18 (air supplying device) is controlled to supply air into the blood circuit. Therefore, it is possible to reduce the amount of the blood to be substituted with the substitution solution (physiological saline solution or dialysate) by substituting the blood with the supplied air. Consequently, it is possible to reliably substitute the blood during returning of the blood and it is possible to suppress the supply amount of the substitution solution. In particular, in the present embodiment, it is not necessary to supply the substitution solution.

Further, the air pump 18 can supply air to or can discharge air from the artery side air trap chamber 5 and the vein side air trap chamber 6. Thus, the liquid surface level inside the artery side air trap chamber 5 and the vein side air trap chamber 6 can be adjusted. Therefore, in addition to the function of supplying the air during returning of the blood after the treatment, it is possible to provide the function of adjusting the liquid surface level in the artery side air trap chamber 5 and the vein side air trap chamber 6 before the treatment or during the treatment.

The present disclosure is not limited to the disclosure. As long as the blood purification apparatus includes the air circulation line connected to the predetermined portion of the blood circuit that can circulate the air and that can supply the air into the blood circuit via the air circulation line during returning of blood, the air pump 18 as the air supplying device may not be provided. However, if the air supplying device (air pump 18) can supply the air into the blood circuit via the air circulation line and the control device can control the air supplying device to supply the air into the blood circuit during returning of the blood, it is possible to more reliably and accurately supply air into the blood circuit during returning of the blood.

In addition, for example, the control device 19 may perform the control during returning of the blood only. Other substitution solutions that are different from the physiological saline solution or the dialysate may be used. Furthermore, in the present embodiment, the artery side air trap chamber 5 and the vein side air trap chamber 6 are respectively connected to the arterial blood circuit 1 and the venous blood circuit 2. However, the present embodiment may be applied to those having the air trap chamber connected to either the arterial blood circuit 1 or the venous blood circuit 2.

Furthermore, in the above-described embodiment, the air circulation line is connected to both the artery side air trap chamber 5 and the vein side air trap chamber 6 to each of which the air can be supplied. However, instead of this manner, the air circulation line may be connected only to either the artery side air trap chamber 5 or the vein side air trap chamber 6. The air may be supplied only to the air trap chamber. In addition, in the present embodiment, the air circulation line is connected to the monitor tubes Lc and Ld. The monitor tubes Lc and Ld are shared in use as a flow route when supplying the air. However, instead of this manner, the air circulation line may be connected to the artery side air trap chamber 5 and the vein side air trap chamber 6 without passing through the monitor tubes Lc and Ld.

The dialysis device B according to the present embodiment is connected to any separate dialysate supplying apparatus that produces the dialysate. Also, it may be applied to a so-called “personal dialysis apparatus” that internally has a function of producing the dialysate. In addition, in the present embodiment, any dialysis device B can be applied to the hemodialysis apparatus. However, it may be applied to a blood purification apparatus that performs other blood purification treatments different from the hemodialysis treatment.

A blood purification apparatus is provided that includes an air circulation line that is connected to a predetermined portion of a blood circuit. Thus, it can circulate air and can supply the air into the blood circuit via the air circulation line during returning of blood. Accordingly, it is possible to apply the blood purification apparatus to apparatuses having a different outer shape or other additional functions.

The present disclosure has been described with reference to the preferred embodiments and modifications. Obviously, other modifications and alternations will occur to those of ordinary skill in the art upon reading and understanding the preceding detailed description. It is intended that the present disclosure be construed to include all such alternations and modifications insofar as they come within the scope of their appended claims or equivalents.

What is claimed is:

1. A blood purification apparatus comprising:
a blood circuit including an arterial blood circuit and a venous blood circuit that extracorporeally circulates blood of a patient from a tip of the arterial blood circuit to a tip of the venous blood circuit;
a blood purification device is interposed between the arterial blood circuit and the venous blood circuit in the blood circuit, the blood purification device purifies the blood flowing in the blood circuit;
the blood purification apparatus can perform returning of blood by substituting the blood inside the blood circuit after treatment;
the blood purification apparatus includes an air circulation line connected to a predetermined portion in the blood circuit, the air circulating line can circulate air; and
during returning of the blood, air can be supplied into the blood circuit via the air circulation line.

2. The blood purification apparatus according to claim 1, further comprising:
an air supplying device supplying air into the blood circuit via the air circulation line; and
a control device controlling the air supplying device to supply the air into the blood circuit during returning of the blood.

3. The blood purification apparatus according to claim 1, further comprising:
an air trap chamber connected to the arterial blood circuit or the venous blood circuit; and
the air can be supplied by connecting the air circulation line to the air trap chamber.

4. The blood purification apparatus according to claim 3, further comprising:
a liquid surface detection sensor to detect a liquid surface of the air trap chamber; and
when the liquid surface sensor detects the liquid surface, the air circulation line stops supplying the air.

5. The blood purification apparatus according to claim 3,
wherein the air supplying device can supply the air to or discharge the air from the air trap chamber, and adjust the liquid surface inside the air trap chamber.

6. The blood purification apparatus according to claim 1,
wherein an air bubble detection sensor detects an air bubble inside a flow route of a tip portion and a valve device opening and closing the flow route of the tip portion, the bubble detection sensor and valve device are arranged in the tip portion of the arterial blood circuit and the venous blood circuit; and
during returning of the blood, when the air bubble detection sensor detects air bubbles, the valve device is in a closed state and the air circulation line stops supplying the air.

7. The blood purification apparatus according to claim 1, further comprising:
a containing device that contains a predetermined amount of a physiological saline solution as a substitution solution; and
a substitution solution supplying device has a physiological saline solution supplying line that supplies the physiological saline solution inside the containing device into the blood circuit by connecting the containing device and a predetermined portion of the blood circuit to each other, and supplying the substitution solution into the blood circuit.

8. The blood purification apparatus according to claim 1,
wherein the blood purification device can back-filtrate a dialysate as the substitution solution and supply the filtered dialysate into the blood circuit.
